(19) United States
(12) Patent Application Publication
(10) Pub. No.: US 2022/0040355 A1
(43) Pub. Date: Feb. 10, 2022

(54) WIRELESS DISINFECTION DEVICE

(71) Applicant: Yi-Chen HSU, Taipei (TW)

(72) Inventor: Yi-Chen HSU, Taipei (TW)

(21) Appl. No.: 17/171,575

(22) Filed: Feb. 9, 2021

(30) Foreign Application Priority Data
Aug. 5, 2020 (TW) 109126548

(51) Int. Cl. A61L 9/14; A61L 2/22

(52) U.S. Cl. CPC A61L 9/14; A61L 2/22; A61L 2202/15; A61L 2209/14; A61L 2209/15; A61L 2209/133; A61L 2209/134; A61L 2202/16

(57) ABSTRACT

A wireless disinfection device, comprising a housing with a grip hole and a through hole. A spraying unit is fitted to the through hole. An air-intake motor, a liquid delivery tube, a nozzle unit and a liquid storage unit are disposed within the housing. A first tube end of the nozzle unit is connected to the spraying unit. A second tube end of the nozzle unit is connected to an air outlet tube of the air-intake motor. A third tube end of the nozzle unit is fitted downwardly to the liquid storage unit. The liquid delivery tube extends from the liquid storage unit and enters into the nozzle unit. The spraying unit includes a nozzle structure. After the air-intake motor is driven, the air can be blown into the nozzle unit such that each tube end of the nozzle unit generates an airflow pressure difference, thereby shooting an disinfecting liquid in the liquid storage unit to a first spray end of the nozzle structure located outside the nozzle orifice. Then, the high-pressure jet is directed to a second spray end of the nozzle structure outside the nozzle orifice such that the air and the disinfecting liquid are combined outside the nozzle orifice to achieve the spray disinfection.

### BACKGROUND OF THE INVENTION

### (a) Field of the Invention

The invention relates to a spraying and disinfecting technology, and more particularly to a wireless disinfection device that can be held in a single hand and achieves a more atomized spraying effect and a better spray disinfection effect in comparison to the conventional sprayers.

### (b) Description of the Related Art

The conventional disinfection sprayer can achieve the spraying effect. However, the atomizing effect and the convenient use thereof are still not ideal, thereby resulting in poor effectiveness of the disinfecting liquid in the conventional disinfection sprayer. Especially, many people currently use spray bottles that can be held with one hand The spray bottle is filled with disinfecting liquid for spray disinfection of the environment. The way of holding the spray bottle with one hand can spray in an atomized manner for disinfection. However, the spraying distance is short. For larger indoor spaces (or outdoors), it will highlight the problem of low spraying efficiency. Moreover, the particle size of the sprayed disinfecting liquid is still large. The disinfection effect of the knapsack sprayer is better. However, if people are burdened with heavy disinfectant buckets, it is even more inconvenient to use. Therefore, how to propose a hand-held disinfection device with both atomized spraying effect and easy-to-use is still a problem to be solved.

### SUMMARY OF THE INVENTION

According to the invention, a wireless disinfection device comprises the following components:

a housing having a grip hole at one end thereof and a through hole at the other end thereof, a spraying unit being disposed in the through hole; and

a first control circuit, a second control circuit, an air-intake motor, a liquid delivery tube, a nozzle unit, a liquid storage unit and a power supply unit coupled to the second control circuit, all of the above-mentioned parts being positioned within the housing;
wherein a first tube end of the nozzle unit is connected to the spraying unit, a second tube end of the nozzle unit is connected to an air outlet tube of the air-intake motor, and a third tube end of the nozzle unit is fitted downwardly to the liquid storage unit, and wherein the liquid delivery tube extends from the liquid storage unit and enters into the nozzle unit, and wherein the internal space of the liquid storage unit, the spraying unit, and the air outlet tube can communicate with each other through both ends of the liquid delivery tube, and wherein the air-intake motor is electrically connected to the first control circuit while the first control circuit is coupled to the second control circuit;
wherein the spraying unit includes a nozzle structure, and the nozzle structure consists of a hollow fin wheel in a sleeve, a screw connection portion protruding from the sleeve, and a water tube holder connected to the screw connection portion, and wherein the other end of the water tube holder is formed with a water guiding tube for connecting the liquid delivery tube, and wherein one end of the sleeve is fixed on the first tube end of the nozzle unit, and the fins of the fin wheel are partially exposed at the other end of the sleeve; and
wherein, after the air-intake motor is driven, the air can be blown into the nozzle unit such that each tube end of the nozzle unit is siphoned, thereby generating an airflow pressure difference; in this way, an disinfecting liquid in the liquid storage unit is sucked into the water guiding tube and delivered to a first spray end of the nozzle structure located outside the nozzle orifice; then, the air is guided to the second spray end of the nozzle structure located outside the nozzle orifice by a flow channel space formed by the fins of the fin wheel such that the air and the disinfecting liquid are combined outside the nozzle orifice to achieve the spray disinfection. Accordingly, the beneficial effects of both atomized spraying and convenient use are achieved.

### BRIEF DESCRIPTION OF THE FIGURES

FIG. 1 is a perspective view of an embodiment of the invention;

FIG. 2 is a perspective exploded view of the embodiment of the invention;

FIG. 3 is a side view of the internal structure of the embodiment of the invention;

FIG. 4 is a perspective view of the internal structure of the embodiment of the invention;

FIG. 5 is a side view of the embodiment of the invention;

FIG. 6 is a partial enlarged schematic view of the spraying unit of the embodiment of the invention;

FIG. 7 is an exploded view of the structure of the spraying unit of the embodiment of the invention; and

FIG. 8 is a perspective view of another embodiment of the invention.

### DETAILED DESCRIPTION OF THE PREFERRED EMBODIMENT

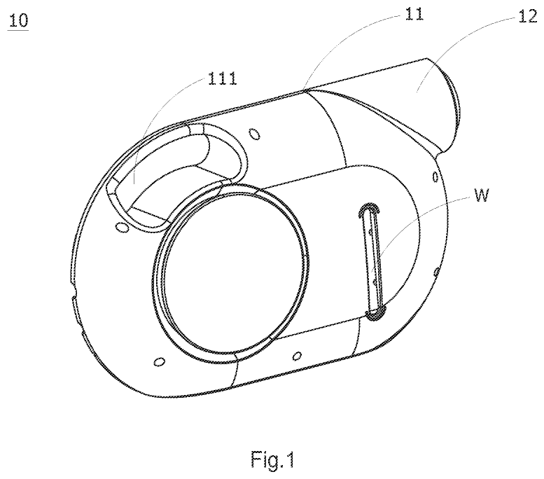

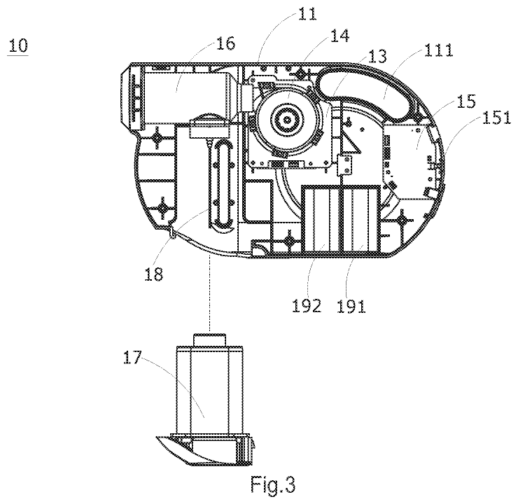

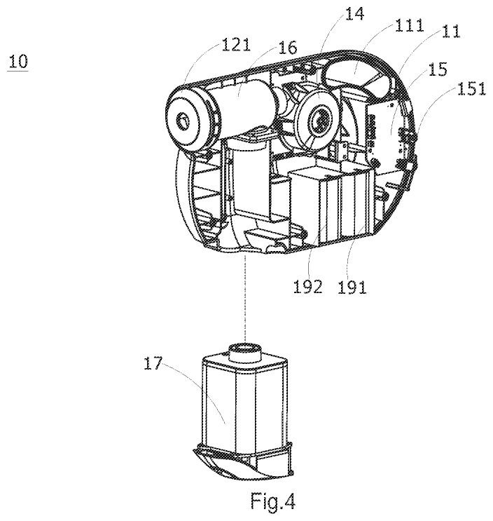

With reference to FIGS. 1 through 7, a wireless disinfection device 10 of the present includes a housing 11 in which a spraying unit 12, a first control circuit 13, an air-intake motor 14, a second control circuit 15, a nozzle unit 16, a liquid storage unit 17, a liquid delivery tube 18 and a power supply unit (not shown) coupled to the second control circuit 15 are provided. The function of each component or the connection relationship between each other is described as follows:

A grip hole 111 is positioned at one end of the housing 11 while a through hole 112 is disposed at the other end thereof. The spraying unit 12 is fitted into the through hole 112. The grip hole 111 allows the user to hold the wireless disinfection device 10 with a single hand, thereby achieving the advantageous effect of being convenient to carry and easy to hold. It can be seen from the drawings that the housing 11 can be mainly composed of an upper cover and a detachable lower cover.

The first control circuit 13 is electrically connected to the air-intake motor 14. The first control circuit 13 is coupled to the second control circuit 15. The aforementioned power supply unit can serve as a power source for the second control circuit 15. The air-intake motor 14 is mainly controlled by the first control circuit 13. The drive of the air-intake motor 14 is indirectly controlled by the second control circuit 15. The reason why two control circuits are proposed in the invention is to avoid the motor-overheating problem caused by higher power components disposed in a concentrated way on the same circuit board (PCB). When the motor is overheated, it may also have a collateral effect on the circuit board, resulting in the overheating problem of the circuit board. As a result, the control circuit (control program) belonging to the air-intake motor 14 is integrated on the first control circuit 13.

A first tube end of the nozzle unit 16 is connected to the spraying unit 12, a second tube end of the nozzle unit 16 is connected to an air outlet tube 141 of the air-intake motor 14, and a third tube end of the nozzle unit 16 is fitted downwardly to the liquid storage unit 17. The liquid delivery tube 18 extends upward from the liquid storage unit 17 and enters into the nozzle unit 16. The part of the liquid delivery tube 18 extending into the nozzle unit 16 is not shown. The internal space of the spraying unit 12, the air outlet tube 141 and the liquid storage unit 17 can communicate with each other through both ends of the liquid delivery tube 18.

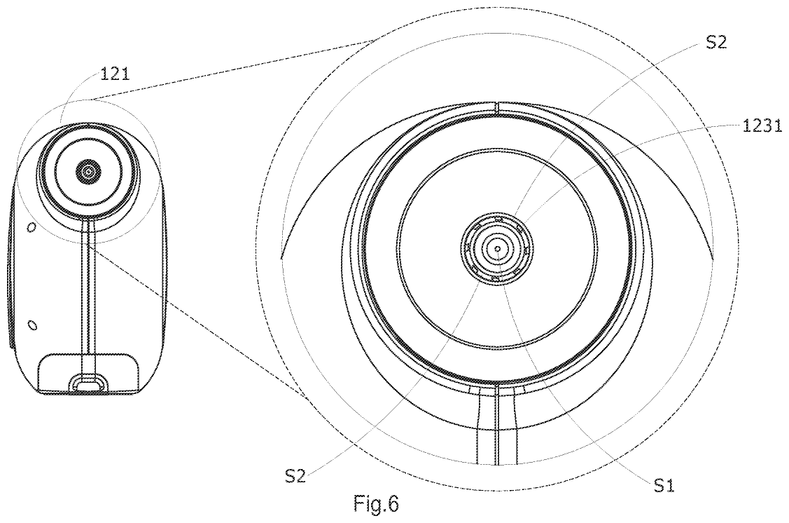

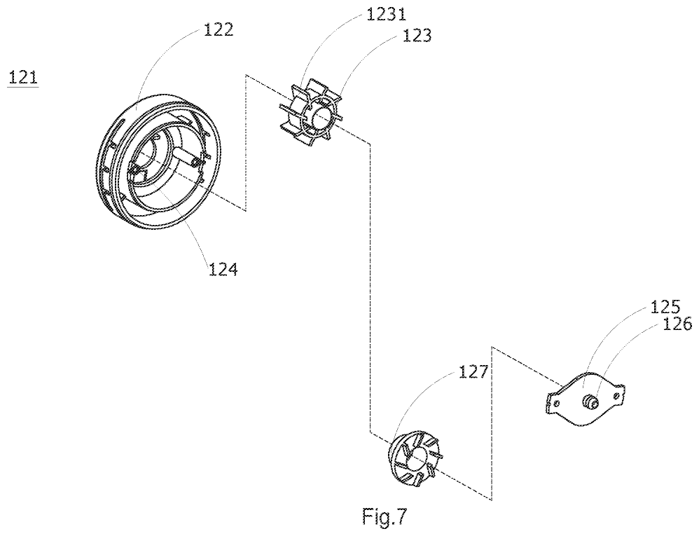

As shown in FIG. 7, the spraying unit 12 includes a nozzle structure 121. The nozzle structure 121 consists of a hollow fin wheel 123 in a sleeve 122, a screw connection portion 124 protruding from the sleeve 122, and a water tube holder 125 connected to the screw connection portion 124. The other end of the water tube holder 125 is formed with a water guiding tube 126. The water guiding tube 126 is used to connect the liquid delivery tube 18. One end of the sleeve 122 is fixed on the first tube end of the nozzle unit 16. The fins 1231 of the fin wheel 123 are partially exposed at the other end of the sleeve 122 (see the second spray end S2 in FIG. 6). Preferably, the nozzle structure 121 may further include a back ring plate 127 stacked on the other end of the fin wheel 123.

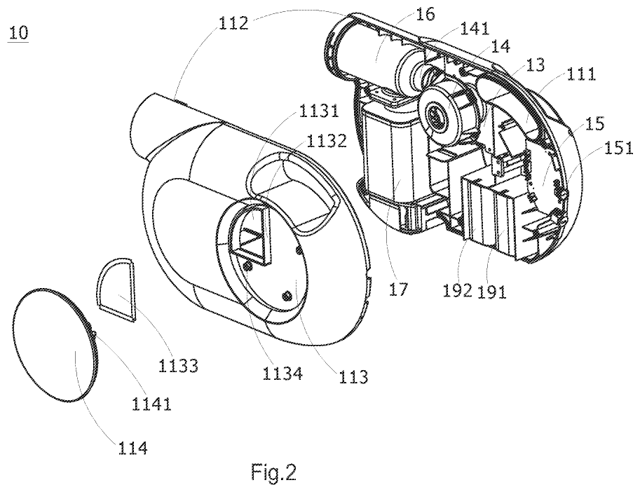

As shown in FIGS. 2 through FIG. 4, the air-intake motor 14 is employed to promote the flow of air. After the air-intake motor 14 is driven, the air can be blown into the nozzle unit 16, so that each tube end of the nozzle unit 16 is siphoned, thereby generating an airflow pressure difference. In this way, an disinfecting liquid in the liquid storage unit 17 is sucked into the water guiding tube 126 and delivered to a first spray end S1 of the nozzle structure 121 located outside the nozzle orifice. The air is guided to the second spray end S2 of the nozzle structure 121 located outside the nozzle orifice by a flow channel space formed by the fins 1231 of the fin wheel 123. As shown in FIG. 6, the second spray end S2 can be a gap between the fins 1231 of the fin wheel 123 partially exposed on the sleeve 1211 such that the air and the disinfecting liquid are combined on the outside of the nozzle orifice. The gap extends from the flow channel space between the two fins. By use of the friction and impact force generated by both air and disinfecting liquid, the disinfecting liquid is separated into fine droplets and atomized to achieve the spray disinfection. Preferably, when the air-intake motor 14 of the wireless disinfection device 10 is activated, the high-pressure jet (air) is first sprayed from the first spray end 51, and the disinfecting liquid is sprayed from the second spray end S2 (that is, the air is discharged first, and then the water is discharged). By use of the high-speed air to crush the disinfecting liquid, the particle size of the disinfecting liquid becomes smaller, which can cause the disinfecting liquid having the smaller particle size to be sprayed, thereby enlarging the contact area of the disinfecting liquid. The reaction efficiency after spraying the disinfecting liquid becomes faster, which means that the disinfecting liquid discharged by the wireless disinfection device of the invention is more atomized than that discharged by the conventional sprayer. While taking into account the spraying effect, the consumption of the disinfecting liquid can also be saved, thereby achieving the full use of the disinfecting liquid.

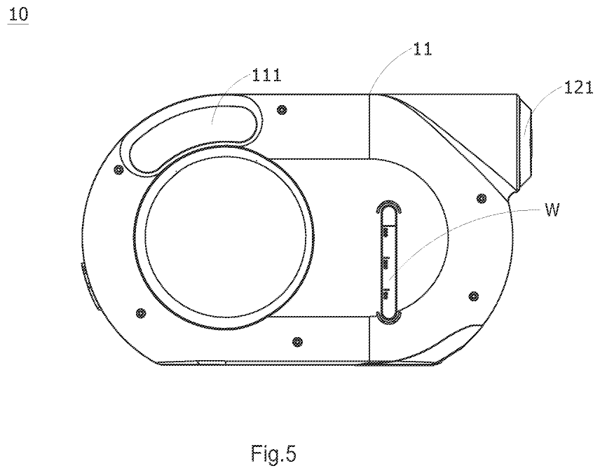

As shown in FIGS. 1 and 5, preferably, a water level window W can be provided at the side surface of the housing 11 relative to the liquid storage unit 17 for observing the level of the disinfecting liquid in the liquid storage unit 17. In this way, it is convenient for the user to determine whether it is necessary to replenish the disinfecting liquid, or whether to replace the bottle of liquid storage unit 17.

As shown in FIGS. 2 through 4, preferably, a sound-absorbing sponge (not shown) can be disposed inside the housing 11 adjacent to the air-intake motor 14 to reduce the noise generated by the air-intake motor 14 when the air-intake motor 14 operates.

Preferably, a first battery compai linent structure 191 for accommodating the power supply unit is positioned within the housing 11 relative to the power supply unit. A second battery compailinent structure 192 is adjacent to the aforementioned first battery compailinent structure 191. The power supply unit can be, for example, one or more lithium batteries. If the power supply unit is composed of two lithium batteries, the aforementioned two battery compailinent structures 191, 192 can respectively house the two lithium batteries of the power supply unit, but should not limited thereto.

According to an embodiment of the invention, the wireless disinfection device 10 may further include a push switch 151 or a touch switch (not shown) half-exposed on the housing 11. The electrical contact of the aforementioned switch can be connected to the control point of the second control circuit 15. In this way, when the aforementioned switch is pressed or touched, the air-intake motor 14 can be driven to spray the disinfecting liquid with strong spray power, so that the wireless disinfection device can atomize the disinfecting liquid more than the conventional sprayer. As a result, the disinfection effect is improved.

According to the embodiment shown in FIGS. 1 through 7 with reference to FIG. 2, the housing 11 of the invention is provided with an air inlet baffle 113 for connecting a circular side plate 114 and for achieving the function of use and the smooth shape of the inlet air flow. An annular oblique gap is formed between the outer edge of the circular side plate 114 and the air inlet baffle 113. The air inlet baffle 113 has a hollow portion 1131 which serves as an air inlet for supplying the air to the air-intake motor 14. The annular oblique gap allows the incoming air to flow into the hollow portion 1131 around the four sides, so that the overall air-intake operation is durable and normal. In addition, the air inlet baffle 113 may be protrudingly provided with a filter retaining wall 1132 adjacent to the hollow portion 1131. Moreover, a filter 1133 is mounted on the inner edge of the filter retaining wall 1132. Preferably, the area of the hollow portion 1131 can occupy a quarter of the area of the air inlet baffle 113 (or the circular side plate 114). The surface of the air inlet baffle 113 may be provided with at least a connecting portion 1134. The circular side plate 114 includes at least a counterpart 1141 engaging into the connecting portion 1134.

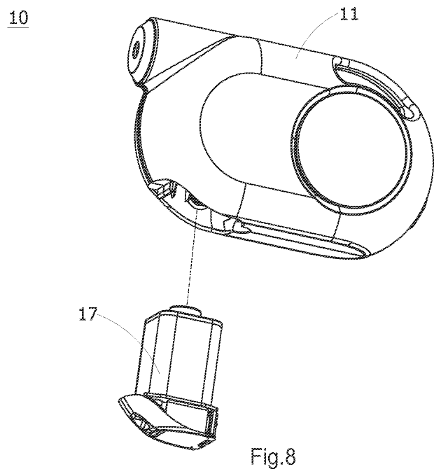

FIG. 8 illustrates another embodiment of the invention. The liquid storage unit 17 of the wireless disinfection device 10 and the outer plate surface of the housing 11 can be fixed by engaging grooves and engaging portions such that the liquid storage unit 17 can be detachable fitted in the housing 11 for the user to take out the liquid storage unit 17. After the disinfecting liquid is replenished, the liquid storage unit 17 may be placed back into the housing 11. Alternatively, another liquid storage unit 17 can be quickly installed after the user takes out the liquid storage unit 17.

What is claimed is:

1. A wireless disinfection device, comprising:
a housing having a grip hole at one end thereof and a through hole at the other end thereof, a spraying unit being disposed in the through hole; and
a first control circuit, a second control circuit, an air-intake motor, a liquid delivery tube, a nozzle unit, a liquid storage unit and a power supply unit coupled to the second control circuit, all of which being positioned within the housing;
wherein a first tube end of the nozzle unit is connected to the spraying unit, a second tube end of the nozzle unit is connected to an air outlet tube of the air-intake motor, and a third tube end of the nozzle unit is fitted downwardly to the liquid storage unit, and wherein the liquid delivery tube extends from the liquid storage unit and enters into the nozzle unit, and wherein the internal space of the liquid storage unit, the spraying unit, and the air outlet tube can communicate with each other through both ends of the liquid delivery tube, and wherein the air-intake motor is electrically connected to the first control circuit while the first control circuit is coupled to the second control circuit;
wherein the spraying unit includes a nozzle structure, and the nozzle structure consists of a hollow fin wheel in a sleeve, a screw connection portion protruding from the sleeve, and a water tube holder connected to the screw connection portion, and wherein the other end of the water tube holder is formed with a water guiding tube for connecting the liquid delivery tube, and wherein one end of the sleeve is fixed on the first tube end of the nozzle unit, and the fins of the fin wheel are partially exposed at the other end of the sleeve; and
wherein, after the air-intake motor is driven, the air can be blown into the nozzle unit such that each tube end of the nozzle unit is siphoned, thereby generating an airflow pressure difference; in this way, an disinfecting liquid in the liquid storage unit is sucked into the water guiding tube and delivered to a first spray end of the nozzle structure located outside the nozzle orifice; then, the air is guided to the second spray end of the nozzle structure located outside the nozzle orifice by a flow channel space formed by the fins of the fin wheel such that the air and the disinfecting liquid are combined outside the nozzle orifice to achieve the spray disinfection.

2. The wireless disinfection device of claim 1, wherein the housing is provided with an air inlet baffle for connecting a circular side plate, and wherein an annular oblique gap is formed between the outer edge of the circular side plate and the air inlet baffle, and wherein the air inlet baffle has a hollow portion which serves as an air inlet for supplying the air to the air-intake motor, and wherein the annular oblique gap allows the incoming air to flow into the hollow portion around the four sides, wherein the air inlet baffle is protrudingly provided with a filter retaining wall adjacent to the hollow portion, and wherein a filter is mounted on the inner edge of the filter retaining wall.

3. The wireless disinfection device of claim 2, wherein the area of the hollow portion occupies a quarter of the area of the air inlet baffle.

4. The wireless disinfection device of claim 2, wherein the surface of the air inlet baffle is provided with at least a connecting portion while the circular side plate includes at least a counterpart engaging into the connecting portion.

5. The wireless disinfection device of claim 1, wherein a sound-absorbing sponge is disposed inside the housing adjacent to the air-intake motor and the air outlet tube to reduce the noise generated by the air-intake motor when the air-intake motor operates.

6. The wireless disinfection device of claim 1, wherein a first battery compartment structure is positioned within the housing relative to the power supply unit while a second battery compailinent structure is adjacent to the first battery compartment structure.

7. The wireless disinfection device of claim 1, wherein a water level window W is provided at the side surface of the housing relative to the liquid storage unit for observing the level of the disinfecting liquid in the liquid storage unit.

8. The wireless disinfection device of claim 1, further comprising a push switch or a touch switch half-exposed on the housing wherein the electrical contact of the switch is connected to the control point of the second control circuit.

9. The wireless disinfection device of claim 1, wherein the liquid storage unit and the outer plate surface of the housing can be fixed by engaging grooves and engaging portions such that the liquid storage unit and the housing are detachably connected.
